(19) United States
(12) Patent Application Publication
(10) Pub. No.: US 2012/0059011 A1
(43) Pub. Date: Mar. 8, 2012

(54) PHARMACEUTICAL COMPOSITIONS OF COMBINATIONS OF DIPEPTIDYL PEPTIDASE-4 INHIBITORS WITH PIOGLITAZONE

(76) Inventors: Nicholas Birringer, Philadelphia, PA (US); Christopher T. John, Perkiomenville, PA (US); Adam Procopio, Cinnaminson, NJ (US); Bhagwant Rege, Collegeville, PA (US); Lawrence A. Rosen, Wayne, PA (US); Sutthilug Sotthivirat, Lansdale, PA (US); Wei Xu, North Wales, PA (US)

(21) Appl. No.: 13/319,718

(22) PCT Filed: Jun. 4, 2010
(86) PCT No.: PCT/US10/37356

§ 371 Date: Nov. 10, 2011

Related U.S. Application Data
(60) Provisional application No. 61/258,958, filed on Nov. 6, 2009.
(60) Provisional application No. 61/187,059, filed on Jun. 15, 2009.

(51) Int. Cl. A61K 31/4439; A61K 31/513; A61P 3/10; A61K 31/4985

(52) U.S. Cl. 514/249; 514/342; 514/274

(57) ABSTRACT

This invention relates to pharmaceutical compositions comprising fixed-dose combinations of a dipeptidyl peptidase-4 inhibitor and pioglitazone, methods of preparing such pharmaceutical compositions, and methods of treating Type 2 diabetes with such pharmaceutical compositions.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral anti-diabetic monotherapy. For many patients, these regimens do not sufficiently control glycaemia during long-term treatment, leading to a requirement for combination therapy within several years following diagnosis. However, co-prescription of two or more oral anti-diabetic drugs may result in treatment regimens that are complex and difficult for many patients to follow. Combining two or more oral anti-diabetic agents into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral anti-diabetic agents include Glucovance™ (metformin and glyburide), Avandamet™ (metformin and rosiglitazone), and Metaglip™ (metformin and glipizide).

Currently sitagliptin phosphate monohydrate and pioglitazone HCl are each available as separate tablets for the treatment of type 2 diabetes. Treatment of type 2 diabetes with the combination of sitagliptin phosphate monohydrate and pioglitazone HCl, acting on different targets, has superior efficacy relative to treatment with either sitagliptin phosphate monohydrate or pioglitazone HCl alone. This invention provides a pharmaceutical composition comprising sitagliptin, or a pharmaceutically acceptable salt thereof, and pioglitazone HCl in a single tablet for superior efficacy in the treatment of type 2 diabetes.

Pioglitazone hydrochloride (ACTOS®) is a thiazolidinedione PPAR-γ agonist used in the management of type 2 diabetes mellitus (also known as non-insulin dependent diabetes mellitus or adult onset diabetes) primarily by decreasing insulin resistance. Pharmacological studies indicate that pioglitazone hydrochloride improves sensitivity to insulin in muscle and adipose tissue, inhibits hepatic gluconeogenesis, and improves glycemic control while reducing circulating insulin levels.

Dipeptidyl peptidase-4 (DPP-4) inhibitors represent a novel class of agents that are being developed for the treatment or improvement in glycemic control in patients with Type 2 diabetes. Specific DPP-4 inhibitors currently in clinical trials for the treatment of Type 2 diabetes include sitagliptin phosphate (MK-0431), vildagliptin (LAF-237), saxagliptin (BMS-47718), alogliptin (X), carmegliptin (X), melogliptin (X), dutogliptin (X), denagliptin (X), linagliptin (X), P93/01 (Prosidion), SYR322 (Takeda), GSK 823093, Roche 0730699, TS021 (Taisho), E3024 (Eisai), and PHX-1149 (Phenomix). For example, oral administration of vildagliptin or sitagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) H.-U. Demuth, et al., “Type 2 diabetes—Therapy with dipeptidyl peptidase IV inhibitors, Biochim. Biophys. Acta, 1751: 33-44 (2005) and (2) K. Augustyns, et al., “Inhibitors of proline-specific dipeptidyl peptidases: DPP IV inhibitors as a novel approach for the treatment of Type 2 diabetes,” Expert Opin. Ther. Patents, 15: 1387-1407 (2005). Sitagliptin phosphate having structural formula I below is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5trifluorophenyl)butan-2-amine.

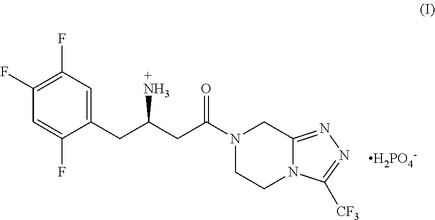

In one embodiment sitagliptin phosphate is in the form of a crystalline anhydrate or monohydrate. In a class of this embodiment, sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in international patent publication WO 2005/0031335 published on. Jan. 13, 2005. For a review on sitagliptin phosphate (MK-0431) including its synthesis and pharmacological properties, reference is made to the following publications: (1) C. F. Deacon, “MK-431,” Curr. Opin. Invest. Drugs, 6: 419-426 (2005) and (2) “MK-0431”, Drugs of the Future,” 30: 337-343 (2005).

Vildagliptin (LAF-237) is the generic name for (S)-1-[(3-hydroxy-1-adamantyl)amino]acetyl-2-cyano-pyrrolidine having structural formula II. Vildagliptin is specifically disclosed in U.S. Pat. No. 6,166,063, the contents of which are hereby incorporated by reference in their entirety.

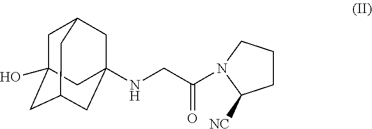

Saxagliptin (BMS-47718) is a methanoprolinenitrile of structural formula III below. Saxagliptin is specifically disclosed in U.S. Pat. No. 6,395,767, the contents of which are hereby incorporated by reference in their entirety.

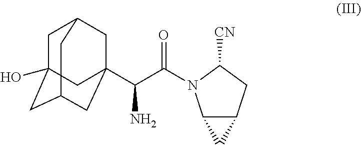

Alogliptin (SYR-322) is a DP-IV inhibitor under investigation for the treatment of type 2 diabetes of structural formula IV below:

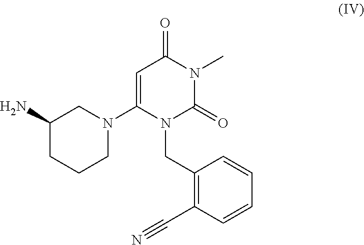

Other DP-IV inhibitors useful in the formulation of the present invention include, but are not limited to: alogliptin, carmegliptin, melogliptin, dutogliptin, denagliptin, linagliptin, saxagliptin and vildagliptin.

The present invention provides for pharmaceutical compositions of a fixed-dose combination of a dipeptidyl peptidase-4 inhibitor (DPP-4 inhibitor) and pioglitazone which are prepared by wet processing methods. The pharmaceutical compositions of the present invention provide for immediate release of the two active pharmaceutical ingredients. In one embodiment the pharmaceutical compositions of the present invention are in the dosage form of a tablet, and, in particular, a film-coated tablet.

The present invention also provides a process to prepare pharmaceutical compositions of a fixed-dose combination of a DPP-4 inhibitor and pioglitazone by wet processing methods. The wet processing methods include wet granulation, such as fluid bed granulation and high-shear granulation.

Another aspect of the present invention provides methods for the treatment of Type 2 diabetes by administering to a host in need of such treatment a therapeutically effective amount of a pharmaceutical composition of the present invention.

These and other aspects will become readily apparent from the detailed description which follows.

### SUMMARY OF THE INVENTION

The present invention is directed to novel pharmaceutical compositions comprising fixed dose combinations of a dipeptidyl peptidase-4 inhibitor (DPP-4 inhibitor) and pioglitazone, or pharmaceutically acceptable salts of each thereof, methods of preparing such pharmaceutical compositions, and methods of treating Type 2 diabetes with such pharmaceutical compositions. In particular, the invention is directed to pharmaceutical compositions comprising fixed-dose combinations of sitagliptin phosphate and pioglitazone hydrochloride.

### DETAILED DESCRIPTION OF THE INVENTION

One aspect of the present invention is directed to dosage forms for the medicinal administration of a fixed-dose combination of a dipeptidyl peptidase-4 inhibitor (DPP-4 inhibitor) and pioglitazone. Such dosage forms may be in the powder or solid format including, but not limited to, tablets, capsules, and sachets. A particular solid dosage form relates to tablets comprising a fixed-dose combination of a DPP-4 inhibitor and pioglitazone hydrochloride (also known as [(±)-5-[[4-[2-(5-ethyl-2-pyridinyl)ethoxy]phenyl]-methyl]-2,4-]thiazolidinedione monohydrochloride).

In a particular aspect of the present invention, the pharmaceutical compositions comprise (a) an intragranular portion comprising: (i) a dipeptidyl peptidase-4 inhibitor, or a pharmaceutically acceptable salt thereof, as one of the two active pharmaceutical ingredients; (ii) pioglitazone hydrochloride as the second active pharmaceutical ingredient; and (iii) a binding agent; and (b) an extragranular portion. In an embodiment of the present invention, the intragranular portion further comprises a disintegrant. In another embodiment of the present invention, the extragranular portion comprises one or more excipients selected from the group consisting of: (a) a diluent; (b) a lubricant; and (c) a disintegrant. In another embodiment of the present invention, the pharmaceutical compositions may also contain one or more surfactants or wetting agents; and one or more antioxidants.

In another embodiment of this aspect of the invention, the DPP-4 inhibitor is selected from the group consisting of sitagliptin, vildagliptin, saxagliptin, P93/01, SYR322, GSK 823093, Roche 0730699, TS021, E3024, and PHX-1149. In a class of this embodiment the DPP-4 inhibitor is alogliptin, carmegliptin, melogliptin, dutogliptin, denagliptin, linagliptin, sitagliptin, vildagliptin, or saxagliptin. In a subclass of this class, the DPP-4 inhibitor is sitagliptin.

A preferred pharmaceutically acceptable salt of sitagliptin is the dihydrogenphosphate salt of structural formula 1 above (sitagliptin phosphate). A preferred form of the dihydrogenphosphate salt is the crystalline monohydrate disclosed in WO 2005/0031335.

The preparation of sitagliptin and pharmaceutically acceptable salts thereof is disclosed in U.S. Pat. No. 6,699,871, the contents of which are herein incorporated by reference in their entirety. The preparation of sitagliptin phosphate monohydrate is disclosed in international patent publication WO 2005/0031335 published on Jan. 13, 2005, the contents of which are herein incorporated by reference in their entirety.

The dosage strength of the DPP-4 inhibitor for incorporation into the pharmaceutical compositions of the present invention is an amount from about 1 milligram to about 250 milligrams of the active moiety. A preferred dosage strength of the DPP-4 inhibitor is an amount from about 25 milligrams to about 200 milligrams of the active moiety. Discrete dosage strengths are the equivalent of 25, 50, 75, 100, 150, and 200 milligrams of the DPP-4 inhibitor active moiety. By “active moiety” is meant the free base form of the DPP-4 inhibitor as an anhydrate.

The unit dosage strength of sitagliptin free base anhydrate (active moiety) for inclusion into the fixed-dose combination pharmaceutical compositions of the present invention is 25, 50, 75, 100, 150, or 200 milligrams. A preferred dosage strength of sitagliptin is 50 or 100 milligrams. An equivalent amount of sitagliptin phosphate monohydrate to the sitagliptin free base anhydrate is used in the pharmaceutical compositions, namely, 32.13, 64.25, 96.38, 128.5, 192.75, and 257 milligrams, respectively.

The dosage strength of pioglitazone for incorporation into the pharmaceutical compositions of the present invention is an amount from about 1 milligram to about 100 milligrams of the active moiety. A preferred dosage strength of pioglitazone is an amount from about 15 milligrams to about 45 milligrams of the active moiety. Discrete dosage strengths are the equivalent of 15, 30, and 45 milligrams of the pioglitazone active moiety. By “active moiety” is meant the free base form of pioglitazone.

The unit dosage strength of the pioglitazone (active moiety) for inclusion into the fixed-dose combination pharmaceutical compositions of the present invention is 15 milligrams, 30 milligrams, and 45 milligrams. An equivalent amount of pioglitazone hydrochloride to the pioglitazone free base (or active moiety) is used in the pharmaceutical compositions, namely, 16.53 milligrams, 33.06 milligrams and 49.59 milligrams, respectively. These unit dosage strengths of pioglitazone represent the dosage strengths approved in the U.S. for marketing to treat Type 2 diabetes.

Specific embodiments of dosage strengths for sitagliptin and pioglitazone in the fixed-dose combinations of the present invention are the following:
(1) 50 milligrams of sitagliptin (equivalent to 64.25 milligrams of sitagliptin phosphate monohydrate) and 15 milligrams pioglitazone (equivalent to 16.53 milligrams of pioglitazone hydrochloride);
(2) 50 milligrams of sitagliptin (equivalent to 64.25 milligrams of sitagliptin phosphate monohydrate) and 30 milligrams pioglitazone (equivalent to 33.06 milligrams of pioglitazone hydrochloride);
(3) 50 milligrams of sitagliptin (equivalent to 64.25 milligrams of sitagliptin phosphate monohydrate) and 45 milligrams pioglitazone (equivalent to 49.59 milligrams of pioglitazone hydrochloride);
(4) 100 milligrams of sitagliptin (equivalent to 128.5 milligrams of sitagliptin phosphate monohydrate) and 15 milligrams pioglitazone (equivalent to 16.53 milligrams of pioglitazone hydrochloride);
(5) 100 milligrams of sitagliptin (equivalent to 128.5 milligrams of sitagliptin phosphate monohydrate) and 30 milligrams pioglitazone (equivalent to 33.06 milligrams of pioglitazone hydrochloride); and
(6) 100 milligrams of sitagliptin (equivalent to 128.5 milligrams of sitagliptin phosphate monohydrate) and 45 milligrams pioglitazone (equivalent to 49.59 milligrams of pioglitazone hydrochloride).

The pharmaceutical compositions of the present invention are prepared by wet processing methods. In one embodiment the pharmaceutical compositions are prepared by wet granulation methods, such as fluid bed granulation or high-shear granulation. In a class of this embodiment, the pharmaceutical compositions are prepared by fluid-bed granulation. Fluid bed granulation processing has the advantage of affording tablets with higher diametric strength. Further, wet processing methods utilizing an intragranular portion containing Sitagliptin phosphate monohydrate, Pioglitazone HCl, a binding agent, and optionally a disintegrant, enhance the chemical stability of Sitagliptin phosphate monohydrate and pioglitazone HCl. In particular, Pioglitazone HCl has been found to react with lubricants, such as magnesium stearate and sodium stearyl fumarate, resulting in disproportionation of the Pioglitazone HCl to the Pioglitazone free base. The fluid bed granulation method utilizing an intragranular portion containing Pioglitazone HCl, Sitagliptin phosphate monohydrate, a binding agent, and optionally a disintegrant, minimizes the disproportionation of Pioglitazone HCl to the pioglitazone free base.

The pharmaceutical compositions obtained by the wet processing methods may be compressed into tablets, encapsulated, or metered into sachets.

The pharmaceutical compositions contain one or more lubricants or glidants. Examples of lubricants include magnesium stearate, calcium stearate, stearic acid, sodium stearyl fumarate, hydrogenated castor oil, and mixtures thereof. In one embodiment, the lubricant is magnesium stearate or sodium stearyl fumarate, or a mixture thereof. In another embodiment, the lubricant is a mixture of magnesium stearate and sodium stearyl fumarate. In another embodiment, the lubricant is magnesium stearate. In another embodiment, the lubricant is sodium stearyl fumarate. Examples of glidants include colloidal silicon dioxide, calcium phosphate tribasic, magnesium silicate, and talc.

The pharmaceutical compositions of the present invention optionally contain one or more binding agents. Embodiments of binding agents include hydroxypropylcellulose (HPC), hydroxypropylmethyl cellulose (HPMC), hydroxyethyl cellulose, starch 1500, polyvinylpyrrolidone (povidone), and co-povidone. In one embodiment, the binding agent is polyvinylpyrrolidone. In another embodiment, the binding agent is hydroxypropylcellulose (HPC). In another embodiment, the binding agent is hydroxypropylcellulose (HPC) in solution. In another embodiment, the binding agent is hydroxypropylcellulose (HPC) in an aqueous solution.

The pharmaceutical compositions of the present invention may also optionally contain one or more diluents. Examples of diluents include mannitol, sorbitol, dibasic calcium phosphate dihydrate, anhydrous dibasic calcium phosphate (also known as anhydrous dicalcium phosphate), microcrystalline cellulose, and powdered cellulose. In one embodiment, the diluent is microcrystalline cellulose. Microcrystalline cellulose is available from several suppliers and includes Avicel PH 101, Avicel PH 102, Avicel, PH 103, Avicel PH 105, and Avicel PH 200, manufactured by the FMC Corporation. In another embodiment, the diluent is mannitol. In another embodiment, the diluent is a mixture of microcrystalline cellulose and mannitol. In another embodiment, the diluent is a 2:1 to 1:2 mixture of microcrystalline cellulose to mannitol. In another embodiment, the diluent is anhydrous dibasic calcium phosphate. Anhydrous dibasic calcium phosphate is available from several suppliers and includes A-TAB™. In another embodiment, the diluent is a mixture of microcrystalline cellulose and anhydrous dibasic calcium phosphate. In another embodiment, the diluent is a 2:1 to 1:2 mixture of microcrystalline cellulose to anhydrous dibasic calcium phosphate. In another embodiment, the diluent is a mixture of mannitol and anhydrous dibasic calcium phosphate.

The pharmaceutical compositions of the present invention may also optionally contain a disintegrant. The disintegrant may be one of several modified starches, modified cellulose polymers, or polycarboxylic acids, such as croscarmellose sodium, sodium starch glycolate, polacrillin potassium, carboxymethylcellulose calcium (CMC Calcium), and crospovidone. In one embodiment, the disintegrant is selected from: polacrillin potassium, carboxymethylcellulose calcium (CMC Calcium), and crospovidone. In another embodiment, the disintegrant is crospovidone.

The pharmaceutical compositions of the present invention may also optionally contain one or more surfactants or wetting agents. The surfactant may be anionic, cationic, or neutral. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral surfactants include glyceryl monooleate, polyoxyethylene sorbitan fatty acid esters, polyvinyl alcohol, and sorbitan esters. Embodiments of wetting agents include poloxamer, polyoxyethylene alkyl ethers, polyoxyethylene castor oil derivatives, and polyoxyethylene stearates.

The pharmaceutical compositions of the present invention may also optionally contain an anti-oxidant which may be added to the formulation to impart chemical stability. The anti-oxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is BHT or BHA.

Preferred dosage forms for the pharmaceutical compositions of the present invention are tablets which are prepared by compression methods. Such tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coating agent is Opadry® which is a formulated powder blend provided by Colorcon. Embodiments of Opadry® useful in the present invention include, but are not limited to, Opadry® I (HPC/HPMC), Opadry® 20A18334, Opadry® II, Opadry® II HP (PVA-PEG), or another suitable Opadry® suspension (such as polyvinyl alcohol, polyethylene glycol, titanium dioxide, and talc, with or without colorants).

Finally, a sweetening agent and/or flavoring agent may be added if desired.

In one embodiment of the present invention, the pharmaceutical composition comprises:
(a) an intragranular portion comprising
   (i) about 15 to 60% by weight of a dipeptidyl peptidase-4 inhibitor, or a pharmaceutically acceptable salt thereof;
   (ii) about 2 to 24% by weight of pioglitazone hydrochloride; and
   (iii) about 1 to 8% by weight of a binding agent; and
(b) an extragranular portion.

In a class of this embodiment, the intragranular portion further comprises about 1 to 8% of a disintegrant. In a subclass of this class, intragranular portion further comprises about 2 to 4% of a disintegrant. In another subclass of this class, intragranular portion further comprises about 2.06 to 3.69% of a disintegrant. In another subclass of this class, intragranular portion further comprises about 2.06 to 2.53% of a disintegrant. In another subclass of this class, the disintegrant is crospovidone.

In another class of this embodiment, the extragranular portion comprises one or more excipients selected from the group consisting of: (a) a diluent; (b) a lubricant; and (c) a disintegrant.

In another class of this embodiment, the extragranular portion comprises one or more excipients selected from the group consisting of: (a) a diluent; (b) a lubricant; and (c) a disintegrant.

In another class of this embodiment, the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate, or a mixture thereof. In another class of this embodiment, the binding agent is hydroxypropylcellulose; the disintegrant is crospovidone; the diluent is microcrystalline cellulose or mannitol, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and mannitol; and the lubricant is sodium stearyl fumarate. In another class of this embodiment, the binding agent is hydroxypropylcellulose; the disintegrant is crospovidone; the diluent is microcrystalline cellulose; and the lubricant is sodium stearyl fumarate.

In another class of this embodiment, the binding agent is hydroxypropylcellulose; the disintegrant is crospovidone; the diluent is a mixture of microcrystalline cellulose and anhydrous dibasic calcium phosphate; and the lubricant is sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a second embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 2 to 24% by weight of pioglitazone hydrochloride; and
   (iii) about 1 to 8% by weight of a binding agent; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the intragranular portion further comprises about 1 to 8% of a disintegrant. In a subclass of this class, intragranular portion further comprises about 2 to 4% of a disintegrant. In another subclass of this class, intragranular portion further comprises about 2.06 to 3.69% of a disintegrant. In another subclass of this class, intragranular portion further comprises about 2.06 to 2.53% of a disintegrant. In another subclass of this class, the disintegrant is crospovidone.

In another class of this embodiment, the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate, or a mixture thereof. In another class of this embodiment, the binding agent is hydroxypropylcellulose; the disintegrant is crospovidone; the diluent is microcrystalline cellulose or mannitol, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and mannitol; and the lubricant is sodium stearyl fumarate. In another class of this embodiment, the binding agent is hydroxypropylcellulose; the disintegrant is crospovidone; the diluent is microcrystalline cellulose; and the lubricant is sodium stearyl fumarate.

In another class of this embodiment, the binding agent is hydroxypropylcellulose; the disintegrant is crospovidone; the diluent is a mixture of microcrystalline cellulose and anhydrous dibasic calcium phosphate; and the lubricant is sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a third embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 2 to 24% by weight of pioglitazone hydrochloride; and
   (iii) about 1 to 8% by weight of a binding agent; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the intragranular portion further comprises about 1 to 8% of a disintegrant. In a subclass of this class, intragranular portion further comprises about 2 to 4% of a disintegrant. In another subclass of this class, intragranular portion further comprises about 2.06 to 3.69% of a disintegrant. In another subclass of this class, intragranular portion further comprises about 2.06 to 2.53% of a disintegrant. In another subclass of this class, the disintegrant is crospovidone.

In another class of this embodiment, the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and mannitol; and the lubricant is sodium stearyl fumarate. In another class of this embodiment, the binding agent is hydroxypropylcellulose; the disintegrant is crospovidone; the diluent is microcrystalline cellulose; and the lubricant is sodium stearyl fumarate.

In another class of this embodiment, the binding agent is hydroxypropylcellulose; the disintegrant is crospovidone; the diluent is a mixture of microcrystalline cellulose and anhydrous dibasic calcium phosphate; and the lubricant is sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a fourth embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 2 to 24% by weight of pioglitazone hydrochloride;
   (iii) about 1 to 8% by weight of a binding agent; and
   (iv) about 1 to 8% by weight of a disintegrant; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate, or a mixture thereof. In another class of this embodiment, the binding agent is hydroxypropylcellulose; the disintegrant is crospovidone; the diluent is microcrystalline cellulose or mannitol, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and mannitol; and the lubricant is sodium stearyl fumarate. In another class of this embodiment, the binding agent is hydroxypropylcellulose; the disintegrant is crospovidone; the diluent is microcrystalline cellulose; and the lubricant is sodium stearyl fumarate.

In another class of this embodiment, the binding agent is hydroxypropylcellulose; the disintegrant is crospovidone; the diluent is microcrystalline cellulose or anhydrous dibasic calcium phosphate, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and anhydrous dibasic calcium phosphate; and the lubricant is sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a fifth embodiment of the present invention, the pharmaceutical composition comprises:
(a) an intragranular portion comprising:
   (1) about 15 to 60% by weight of a dipeptidyl peptidase-4 inhibitor, or a pharmaceutically acceptable salt thereof;
   (ii) about 2 to 24% by weight of pioglitazone hydrochloride;
   (iii) about 1 to 8% by weight of a binding agent; and
   (iv) about 1 to 8% by weight of a disintegrant; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate. In another class of this embodiment, the binding agent is hydroxypropylcellulose; the disintegrant is crospovidone; the diluent is a mixture of microcrystalline cellulose and mannitol; and the lubricant is sodium stearyl fumarate. In another class of this embodiment, the binding agent is hydroxypropylcellulose; the disintegrant is crospovidone; the diluent is microcrystalline cellulose; and the lubricant is sodium stearyl fumarate.

In another class of this embodiment, the binding agent is hydroxypropylcellulose; the disintegrant is crospovidone; the diluent is a mixture of microcrystalline cellulose and anhydrous dibasic calcium phosphate; and the lubricant is sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a sixth embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 2 to 24% by weight of pioglitazone hydrochloride;
   (iii) about 1 to 10% by weight of a binding agent; and
   (iv) about 1 to 8% of a disintegrant; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a seventh embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 2 to 24% by weight of pioglitazone hydrochloride;
   (iii) about 1 to 8% by weight of a binding agent; and
   (iv) about 1 to 8% of a disintegrant; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In an eighth embodiment of the present invention, the pharmaceutical composition comprises:
(a) an intragranular portion comprising:
   (i) about 21 to 33% by weight of a dipeptidyl peptidase-4 inhibitor, or a pharmaceutically acceptable salt thereof;
   (ii) about 4 to 13% by weight of pioglitazone hydrochloride;
   (iii) about 2 to 6% by weight of a binding agent; and
   (iv) about 2 to 4% of a disintegrant; and
(b) an extragranular portion comprising:
   (i) about 2 to 5% by weight of a disintegrant;
   (ii) about 44 to 54% by weight of a diluent; and
   (iii) about 0.5 to 4% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a ninth embodiment of the present invention, the pharmaceutical composition comprises:
(a) an intragranular portion comprising:
   (i) about 21.42 to 32.13% by weight of a dipeptidyl peptidase-4 inhibitor, or a pharmaceutically acceptable salt thereof;
   (ii) about 4.13 to 12.4% by weight of pioglitazone hydrochloride;
   (iii) about 2.88 to 5.16% by weight of a binding agent; and
   (iv) about 2.06 to 3.69 % of a disintegrant; and
(b) an extragranular portion comprising:
   (i) about 3% by weight of a disintegrant;
   (ii) about 44.4 to 53.8% by weight of a diluent; and
   (iii) about 1 to 2 % by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a tenth embodiment of the present invention, the pharmaceutical composition comprises:
(a) an intragranular portion comprising:
   (i) about 21 to 33% by weight of a dipeptidyl peptidase-4 inhibitor, or a pharmaceutically acceptable salt thereof;
   (ii) about 3 to 13% by weight of pioglitazone hydrochloride;
   (iii) about 2 to 6% by weight of a binding agent; and
   (iv) about 2 to 6% of a disintegrant; and
(b) an extragranular portion comprising:
   (i) about 2 to 6% by weight of a disintegrant;
   (ii) about 40 to 55% by weight of a diluent; and
   (iii) about 0.5 to 4% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In an eleventh embodiment of the present invention, the pharmaceutical composition comprises:
(a) an intragranular portion comprising:
   (i) about 15 to 60% by weight of a dipeptidyl peptidase-4 inhibitor, or a pharmaceutically acceptable salt thereof;
   (ii) about 4 to 15% by weight of pioglitazone hydrochloride;
   (iii) about 1 to 8% by weight of a binding agent; and
   (iv) about 1 to 8% of a disintegrant; and
(b) an extragranular portion comprising:
   (i) about 2 to 9% by weight of a disintegrant;
   (ii) about 0 to 80% by weight of a diluent; and
   (iii) about 0.1 to 10% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a twelfth embodiment of the present invention, the pharmaceutical composition comprises:
(a) an intragranular portion comprising:
   (i) about 21 to 33% by weight of a dipeptidyl peptidase-4 inhibitor, or a pharmaceutically acceptable salt thereof;
   (ii) about 4 to 13.5% by weight of pioglitazone hydrochloride;
   (iii) about 2 to 6% by weight of a binding agent; and
   (iv) about 2 to 4% of a disintegrant; and
(b) an extragranular portion comprising:
   (i) about 2 to 5% by weight of a disintegrant;
   (ii) about 44 to 55% by weight of a diluent; and
   (iii) about 0.5 to 4% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In a thirteenth embodiment of the present invention, the pharmaceutical composition comprises:
(a) an intragranular portion comprising:
   (i) about 25.7 to 32.13% by weight of a dipeptidyl peptidase-4 inhibitor, or a pharmaceutically acceptable salt thereof;
   (ii) about 4.13 to 13.22% by weight of pioglitazone hydrochloride;
   (iii) about 2.88 to 3.54% by weight of a binding agent; and
   (iv) about 2.06 to 2.53% of a disintegrant; and
(b) an extragranular portion comprising:
   (i) about 3% by weight of a disintegrant;
   (ii) about 44.91 to 54.3% by weight of a diluent; and
   (iii) about 1 to 2% by weight of a lubricant.

In a class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and mannitol; and the disintegrant is crospovidone.

In another class of this embodiment, the dipeptidyl peptidase-4 inhibitor is sitagliptin, or a pharmaceutically acceptable salt thereof; the lubricant is magnesium stearate; the binding agent is hydroxypropylcellulose; the diluent is a mixture of microcrystalline cellulose and anhydrous dibasic calcium phosphate; and the disintegrant is crospovidone.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogenphosphate salt thereof.

In another class of the embodiments of the present invention, the pharmaceutical composition contains about 15 to 60% by weight of sitagliptin phosphate. In a subclass of this class, the composition contains about 21 to 33% of sitagliptin phosphate. In another subclass of this class, the composition contains about 21.42 to 32.13% of sitagliptin phosphate. In another subclass of this class, the composition contains about 25.7 to 32.13% of sitagliptin phosphate.

In another class of the embodiments of the present invention, the pharmaceutical composition contains about 15 to 60% by weight of sitagliptin, or a pharmaceutically acceptable salt thereof. In a subclass of this class, the composition contains about 21 to 33% of sitagliptin, or a pharmaceutically acceptable salt thereof. In another subclass of this class, the composition contains about 21.42 to 32.13% of sitagliptin, or a pharmaceutically acceptable salt thereof. In another subclass of this class, the composition contains about 25.7 to 32.13% of sitagliptin, or a pharmaceutically acceptable salt thereof. In another subclass of this class, the composition contains about 31% of sitagliptin, or a pharmaceutically acceptable salt thereof.

In another class of the embodiments of the present invention, the pharmaceutical composition contains about 2 to 24% by weight of pioglitazone HCl. In a subclass of this class, the composition contains about 4 to 13% of pioglitazone HCl. In another subclass of this class, the composition contains about 4.13 to 12.4% of pioglitazone HCl. In another subclass of this class, the composition contains about 4 to 4.5% of pioglitazone HCl. In another subclass of this class, the composition contains about 8 to 8.5% of pioglitazone HCl. In another subclass of this class, the composition contains about 10.75 to 11.25% of pioglitazone HCl. In another subclass of this class, the composition contains about 12 to 12.5% of pioglitazone HCl. In another subclass of this class, the composition contains about 4 to 15% of pioglitazone HCl. In another subclass of this class, the composition contains about 4 to 13.5% of pioglitazone HCl. In another subclass of this class, the composition contains about 4.13 to 13.22% of pioglitazone HCl.

In another class of the embodiments of the present invention, the pharmaceutical composition contains about 2 to 24% by weight of pioglitazone, or a pharmaceutically acceptable salt thereof. In a subclass of this class, the composition contains about 4 to 13% of pioglitazone, or a pharmaceutically acceptable salt thereof. In another subclass of this class, the composition contains about 4,13 to 12.4% of pioglitazone, or a pharmaceutically acceptable salt thereof. In another subclass of this class, the composition contains about 4 to 4.5% of pioglitazone, or a pharmaceutically acceptable salt thereof. In another subclass of this class, the composition contains about 8 to 8.5% of pioglitazone, or a pharmaceutically acceptable salt thereof. In another subclass of this class, the composition contains about 10.75 to 11.25% of pioglitazone, or a pharmaceutically acceptable salt thereof. In another subclass of this class, the composition contains about 12 to 12.5% of pioglitazone, or a pharmaceutically acceptable salt thereof. In another subclass of this class, the composition contains about 4 to 15% of pioglitazone, or a pharmaceutically acceptable salt thereof. In another subclass of this class, the composition contains about 4 to 13.5% of pioglitazone, or a pharmaceutically acceptable salt thereof. In another subclass of this class, the composition contains about 4.13 to 13.22% of pioglitazone, or a pharmaceutically acceptable salt thereof.

In another class of the embodiments of the present invention, the pharmaceutical composition contains about 0 to 80% by weight of a diluent. In a subclass of this class, the composition contains about 40 to 55% of a diluent. In another subclass of this class, the composition contains about 43 to 55% of a diluent. In another subclass of this class, the composition contains about 44 to 55% of a diluent. In another subclass of this class, the composition contains about 44 to 54% of a diluent. In another subclass of this class, the composition contains about 44.4 to 53.8% of a diluent. In another subclass of this class, the composition contains about 44.91 to 54.3% of a diluent. In another subclass of this class, the composition contains about 30 to 31% of a first diluent and 13 to 24% of a second diluent. In another subclass of this class, the composition contains about 22 to 27.5% of a first diluent and 22 to 27.5% of a second diluent. In another subclass of this class, the diluent is microcrystalline cellulose, mannitol or anhydrous dibasic calcium phosphate. In another subclass of this class, the diluent is a mixture of microcrystalline cellulose and mannitol. In another subclass of this class, the diluent is a 1:1 mixture of microcrystalline cellulose and mannitol. In another subclass of this class, the diluent is a mixture of microcrystalline cellulose and anhydrous dibasic calcium phosphate. In another subclass of this class, the diluent is a 1:1 mixture of microcrystalline cellulose and anhydrous dibasic calcium phosphate. In another subclass of this class, the diluent is microcrystalline cellulose. In another subclass of this class, the diluent is mannitol. In another subclass of this class, the diluent is anhydrous dibasic calcium phosphate.

In another class of the embodiments of the present invention, the intragranular portion of the pharmaceutical composition optionally contains about 1 to 8% by weight of a disintegrant. In a subclass of this class, the intragranular portion of the composition optionally contains about 2 to 6% of a disintegrant. In another subclass of this class, the intragranular portion of the composition optionally contains about 2 to 4% of a disintegrant. In another subclass of this class, the intragranular portion of the composition optionally contains about 2.06 to 3.69% of a disintegrant. In another subclass of this class, intragranular portion of the composition optionally contains about 2.06 to 2.53% of a disintegrant. In another subclass of this class, the disintegrant is crospovidone.

In another class of the embodiments of the present invention, the extragranular portion of the pharmaceutical composition contains about 2 to 9% by weight of a disintegrant. In another subclass of this class, the extragranular portion of the composition contains about 2 to 5% of a disintegrant. In another subclass of this class, the extragranular portion of the composition contains about 3% of a disintegrant. In another subclass of this class, the disintegrant is crospovidone.

In another class of the embodiments of the present invention, the pharmaceutical composition contains about 0.1 to 10% by weight of a lubricant. In a subclass of this class, the composition contains about 0.5 to 4% of a lubricant. In another subclass of this class, the composition contains about 1 to 2% of a lubricant. In another subclass of this class, the composition contains about 1.5% of a lubricant. In another subclass of this class, the composition contains about 2% of a lubricant. In another subclass of this class, the lubricant is sodium stearyl fumarate or magnesium stearate. In another subclass of this class, the lubricant is sodium stearyl fumarate and magnesium stearate. In another subclass of this class, the lubricant is sodium stearyl fumarate. In another subclass of this class, the lubricant is magnesium stearate. In another class of this embodiment, the binding agent is hydroxypropylcellulose or polyvinylpyrrolidone, and the lubricant is sodium stearyl fumarate or magnesium stearate. In another class of this embodiment, the binding agent is hydroxypropylcellulose, and the lubricant is sodium stearyl fumarate. In another class of this embodiment, the binding agent is hydroxypropylcellulose, and the lubricant is magnesium stearate.

In another class of the embodiments of the present invention, the pharmaceutical composition contains about 1 to 10% by weight of a binding agent. In a subclass of this class, the composition contains about 1 to 8% of a binding agent. In another subclass of this class, the composition contains about 2 to 7% of a binding agent. In another subclass of this class, the composition contains about 2 to 6% of a binding agent. In another subclass of this class, the composition contains about 2.8 to 5.2% of a binding agent. In another subclass of this class, the composition contains about 2.88 to 5.16% of a binding agent. In another subclass of this class, the composition contains about 2.88 to 3.54% of a binding agent. In another subclass of this class, the binding agent is hydroxypropylcellulose or polyvinylpyrrolidone, and the lubricant is sodium stearyl fumarate or magnesium stearate. In another subclass of this class, the binding agent is hydroxypropylcellulose, and the lubricant is sodium stearyl fumarate. In another subclass of this class, the binding agent is hydroxypropylcellulose, and the lubricant is magnesium stearate.

In further embodiments of the present invention, the pharmaceutical compositions are envisioned for commercial development:
(A) Tablets of 50 mg dipeptidyl peptidase-4 inhibitor/15 mg pioglitazone potency. About 8.27% by weight of the pioglitazone hydrochloride, or a pharmaceutically acceptable salt thereof; about 32.13% by weight of the dipeptidyl peptidase-4 inhibitor; about 3.21% by weight of a binding agent; about 49.1-49.6% by weight of a diluent; about 1 to 2% by weight of a lubricant; and about 5.29% by weight of a disintegrant. In a class of this embodiment the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of this embodiment the dipeptidyl peptidase-4 inhibitor is sitagliptin, vildagliptin, or saxagliptin; the binding agent is hydroxypropyl-cellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin.
(B) Tablets of 50 mg dipeptidyl peptidase-4 inhibitor/30 mg pioglitazone potency. About 11.02% by weight of the pioglitazone hydrochloride, or a pharmaceutically acceptable salt thereof; about 21.42% by weight of the dipeptidyl peptidase-4 inhibitor; about 5.16% by weight of a binding agent; about 53.72-54.22% by weight of a diluent; about 1 to 2% by weight of a lubricant; and about 6.69% by weight of a disintegrant. In a class of this embodiment the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of this embodiment the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin. Alternatively, about 13.22% by weight of the pioglitazone hydrochloride, or a pharmaceutically acceptable salt thereof; about 25.70% by weight of the dipeptidyl peptidase-4 inhibitor; about 3.1% by weight of a binding agent; about 50.26-52.26% by weight of a diluent; about 1 to 2% by weight of a lubricant; and about 5.21% by weight of a disintegrant. In a class of this embodiment the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of this embodiment the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin.
(C) Tablets of 100 mg dipeptidyl peptidase-4 inhibitor/15 mg pioglitazone potency. About 4.13% by weight of the pioglitazone hydrochloride, or a pharmaceutically acceptable salt thereof; about 32.13% by weight of the dipeptidyl peptidase-4 inhibitor; about 2.88% by weight of a binding agent; about 53.8-54.30% by weight of a diluent; about 1 to 2% by weight of a lubricant; and about 5.06% by weight of a disintegrant. In a class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin.
(D) Tablets of 100 mg Dipeptidyl peptidase-4 inhibitor/30 mg pioglitazone potency. About 8.27% by weight of the pioglitazone hydrochloride, or a pharmaceutically acceptable salt thereof; about 32.13% by weight of the dipeptidyl peptidase-4 inhibitor; about 3.21% by weight of a binding agent; about 49.1-49.6% by weight of a diluent; about 1 to 2% by weight of a lubricant; and about 5.29% by weight of a disintegrant. In a class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin.
(E) Tablets of 100 mg dipeptidyl peptidase-4 inhibitor/45 mg pioglitazone potency. About 12.4% by weight of the pioglitazone hydrochloride, or a pharmaceutically acceptable salt thereof; about 32.13% by weight of the dipeptidyl peptidase-4 inhibitor; about 3.54% by weight of a binding agent; about 44.4-44.9% by weight of a diluent; about 1 to 2% by weight of a lubricant; and about 5.53% by weight of a disintegrant. In a class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or mannitol or a mixture thereof, and the optional disintegrant is crospovidone. In a subclass of this class, the dipeptidyl peptidase-4 inhibitor is sitagliptin. In another class of these embodiments the dipeptidyl peptidase-4 inhibitor is sitagliptin, vildagliptin, or saxagliptin; the binding agent is hydroxypropylcellulose, the lubricant is magnesium stearate or sodium stearyl fumarate, the diluent is microcrystalline cellulose or anhydrous dibasic calcium phosphate, or a mixture thereof; and the optional disintegrant is crospovidone. In a subclass of this class, the dipeptidyl peptidase-4 inhibitor is sitagliptin.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated. Substances which may be used for coating include hydroxypropylcellulose, hydroxypropylmethylcellulose, titanium dioxide, talc, sweeteners, colorants, and flavoring agents.

The terms and symbols “% by weight” and “%” as used herein refer to the percentage by weight of the total tablet weight, wherein the term “total tablet weight” includes both the weight of the intragranular and extragranular portions but excludes the weight of the coating.

The term “or a mixture thereof' as used herein refers to a mixture of two excipients selected from the group of excipients, or three excipients selected from the group of excipients, or more than three excipients selected from the group of excipients.

In one embodiment the pharmaceutical compositions of the present invention are prepared by wet granulation (high shear and/or fluid bed). In one class of this embodiment, the pharmaceutical composition was prepared by fluid bed wet granulation. In another class of this embodiment, the pharmaceutical composition was prepared by high shear wet granulation. Granulation is a process in which binding agent is added either through the granulating solution or through addition to the granulating bowl to form granules. The steps involved in the wet granulation method comprise the following:
(1) the active pharmaceutical ingredients pioglitazone hydrochloride and the DPP-4 inhibitor, or a pharmaceutically acceptable salt thereof, are added to the granulating bowl;
(2) optional disintegrant(s) are added to step 1;
(3) for high shear granulation, the binding agent (such as polyvinylpyrrolidone or hydroxypropylcellulose) is added dry to the granulating bowl and dry mixed for a short period followed by the addition of water with or without a surfactant (such as sodium lauryl sulfate). For fluid bed granulation, both active pharmaceutical ingredients are added to the granulator bowl and the granulating solution comprised of binding agent, such as hydroxypropylcellulose, in water, with or without a surfactant, is added upon fluidization;
(4) granules prepared by high shear granulation are tray-dried in an oven or dried in a fluid bed dryer. For granules prepared by fluid bed granulation, granules are dried in a fluid bed dryer;
(5) dried granules are resized in suitable mill;
(6) optional diluent (such as microcrystalline cellulose and/or mannitol and/or anhydrous dibasic calcium phosphate) is blended with dried granules in a suitable blender;
(7) optional disintegrant (such as crospovidone) is added to step 6;
(8) lubricants or glidants (such as magnesium stearate and sodium stearyl fumarate) are added to the blend from step 5, 6 or 7 in a suitable blender;
(9) lubricated granule mixture from step 8 may be filled into bottles, sachets, or capsules or compressed into desired tablet image;
(10) and optionally, the resulting tablets may be film-coated.

A suitable wet granulation method comprises the following steps:
(1) a binding agent solution is prepared by dissolving binding agent hydroxypropylcellulose (HPC) in water;
(2) sitagliptin phosphate and pioglitazone HCl along with optional disintegrant crospovidone are charged into a fluid bed granulator and granulated with the aqueous HPC solution;
(3) the granules are dried;
(4) the dried granules are de-lumped using suitable milling equipment;
(5) the granules are blended with extragranular filler (microcrystalline cellulose or mannitol or anhydrous dibasic calcium phosphate, or microcrystalline cellulose and mannitol, or microcrystalline cellulose and anhydrous dibasic calcium phosphate) and disintegrant crospovidone in a suitable blender for 10 minutes;
(6) the blended granules are lubricated with magnesium stearate or sodium stearyl fumarate in a suitable blender for 5 minutes;
(7) the final blend is tableted in a suitable tableting machine; and
(8) the tablets are coated with Opadry coating system to desired weight gain.

The present invention provides a fixed dose combination of a dipeptidyl peptidase-4 inhibitor, or a pharmaceutically acceptable salt thereof, and pioglitazone, or a pharmaceutically acceptable salt thereof, in which both drugs are stable in a single tablet. More particularly, the present invention provides a fixed dose combination of a dipeptidyl peptidase-4 inhibitor, or a pharmaceutically acceptable salt thereof, and pioglitazone HCl in a single tablet, in which the conversion of pioglitazone HCl to the pioglitazone free base via disproportionation is minimized. The present invention also provides methods for treating Type 2 diabetes by orally administering to a host in need of such treatment a therapeutically effective amount of one of the fixed-dose combination pharmaceutical compositions of the present invention. In one embodiment the host in need of such treatment is a human. In another embodiment the pharmaceutical composition is in the dosage form of a tablet. The pharmaceutical compositions comprising the fixed-dose combination may be administered once-daily (QD), twice-daily (BID), or thrice-daily (TID). The following examples further describe and demonstrate embodiments within the scope of the present invention. The examples are given solely for the purpose of illustration and are not intended to be construed as limitations of the present invention as many variations thereof are possible without departing from the spirit and scope of the invention.

### EXAMPLE 1

Fixed-dose combination of 100 milligrams sitagliptin and 15 milligrams
pioglitazone/per tablet - fluid bed wet granulation

| Ingredient (mg/unit) | 100 mg/15 mg |
|---|---|
| Sitagliptin phosphate monohydrate* | 128.5 mg |
| Pioglitazone hydrochloride** (jet milled) | 16.53 mg |
| Hydroxypropylcellulose (HPC) | 11.54 mg |
| Microcrystalline cellulose (Avicel PH-102) | 108.60 mg |
| Mannitol (SD 200) | 108.60 mg |
| Crospovidone | 20.24 mg |
| 12 mg extragranular; 8.24 mg intragranular | |
| Sodium stearyl fumarate | 6.00 mg |
| Purified water for granulation step*** | 180.79 mg containing |
| | the above 11.54 mg of |
| | HPC for fluid bed |
| Total Tablet Weight | 400 mg |
| Opadry ® 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 15 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose was added to the mixture of sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After milling/de-lumping, microcrystalline cellulose, mannitol, and crospovidone were added to the granules and blended in a twin shell-blender for 10 minutes. Then the lubricant sodium stearyl fumarate was added, and the mixture was blended an additional 5 minutes. The resulting lubricated mixture was compressed using a rotary tablet press to provide a 400 mg uncoated tablet. The tablets were optionally film coated with a suitable Opadry® suspension (such as Opadry® 20A18334) to an approximate 3% weight gain to provide a 412 mg coated tablet.

### EXAMPLE 2

Fixed-dose combination of 100 milligrams sitagliptin and 45 milligrams
pioglitazone/per tablet - fluid bed wet granulation

| Ingredient (mg/unit) | 100 mg/45 mg |
|---|---|
| Sitagliptin phosphate monohydrate* | 128.5 mg |
| Pioglitazone hydrochloride** (jet milled) | 49.59 mg |
| Hydroxypropylcellulose (HPC) | 14.17 mg |
| Microcrystalline cellulose (Avicel PH-102) | 89.81 mg |
| Mannitol (SD 200) | 89.81 mg |
| Crospovidone | 22.12 mg |
| 12 mg extragranular; 10.12 mg intragranular | |
| Sodium stearyl fumarate | 6.00 mg |
| Purified water for granulation step*** | 221.99 mg containing |
| | the above 14.17 mg of |
| | HPC for fluid bed |
| Total Tablet Weight | 400 mg |
| Opadry ® 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 45 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to the mixture of sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After milling/de-lumping, microcrystalline cellulose, mannitol and crospovidone were added to the granules, and blended in a twin shell-blender for 10 minutes. The lubricant sodium stearyl fumarate was then added, and the mixture was blended an additional 5 minutes. Then the lubricated mixture was compressed using a rotary tablet press to provide a 400 mg uncoated tablet. The tablets were optionally film coated with a suitable Opadry® suspension (such as Opadry® 20A18334) to an approximate 3% weight gain to provide a 412 mg coated tablet.

### EXAMPLE 3

Fixed-dose combination of 100 milligrams sitagliptin and 45 milligrams
pioglitazone/per tablet - fluid bed wet granulation

| Ingredient (mg/unit) | 100 mg/45 mg |
|---|---|
| Sitagliptin phosphate monohydrate* | 128.5 mg |
| Pioglitazone hydrochloride** (jet milled) | 49.59 mg |
| Hydroxypropylcellulose (HPC) | 14.17 mg |
| Microcrystalline cellulose (Avicel PH-102) | 179.62 mg |
| Crospovidone | 22.12 mg |
| 12 mg extragranular; 10.12 mg intragranular | |
| Sodium stearyl fumarate | 6.00 mg |
| Purified water for granulation step*** | 221.99 mg containing |
| | the above 14.17 mg of |
| | HPC mg for fluid bed |
| Total Tablet Weight | 400 mg |
| Opadry ® 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 45 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After milling/de-lumping, microcrystalline cellulose and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 400 mg uncoated tablet. The tablets were optionally film coated with a suitable Opadry® suspension (such as Opadry 20A18334) to an approximate 3% weight gain to provide a 412 mg coated tablet.

### EXAMPLE 4

Fixed-dose combination of 100 milligrams sitagliptin and 30 milligrams
pioglitazone/per tablet - fluid bed wet granulation

| Ingredient (mg/unit) | 100 mg/30 mg |
|---|---|
| Sitagliptin phosphate monohydrate* | 128.5 mg |
| Pioglitazone hydrochloride** (jet milled) | 33.06 mg |
| Hydroxypropylcellulose (HPC) | 12.85 mg |
| Microcrystalline cellulose (Avicel PH-102) | 99.20 mg |
| Mannitol (SD 200) | 99.20 mg |
| Crospovidone | 21.18 mg |
| 12 mg extragranular; 9.18 mg intragranular | |
| Sodium stearyl fumarate | 6.00 mg |
| Purified water for granulation step*** | 201.32 mg containing |
| | the above 12.85 mg of |
| | HPC for fluid bed |
| Total Tablet Weight | 400 mg |
| Opadry ® 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 45 mg of pioglitazone free base.
***Removed during processing.

Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to the mixture of sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, mannitol and crospovidone were added to the granules, and blended in a twin shell-blender for 10 minutes. The lubricant sodium stearyl fumarate was then added, and the mixture was blended an additional 5 minutes. Then the lubricated mixture was compressed using a rotary tablet press to provide a 400 mg uncoated tablet. The tablets were optionally film coated with a suitable Opadry® suspension (such as Opadry®20A18334) to an approximate 3% weight gain to provide a 412 mg coated tablet.

### EXAMPLE 5

Fixed-dose combination of 100 milligrams sitagliptin and 15 milligrams
pioglitazone/per tablet - fluid bed wet granulation

| Ingredient (mg/unit) | 100 mg/15 mg |
|---|---|
| Sitagliptin phosphate monohydrate* | 128.5 mg |
| Pioglitazone hydrochloride** (jet milled) | 16.53 mg |
| Hydroxypropylcellulose (HPC) | 11.54 mg |
| Microcrystalline cellulose (Avicel PH-102) | 93.44 mg |
| Dibasic Calcium Phosphate, anhydrous (A-Tab) | 123.75 mg |
| Crospovidone | 20.24 mg |
| 12 mg extragranular; 8.24 mg intragranular | |
| Sodium stearyl fumarate | 6.00 mg |
| Purified water for granulation step*** | 180.79 mg containing |
| | the above 11.54 mg of |
| | HPC for fluid bed |
| Total Tablet Weight | 400 mg |
| Opadry ® White 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 15 mg of pioglitazone free base.
***Removed during processing.

Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, anhydrous dibasic calcium phosphate and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 400 mg uncoated tablet. The tablets were optionally film coated with a suitable Opadry® suspension (such as Opadry® 20A18334) to an approximate 3% weight gain to provide a 412 mg coated tablet.

### EXAMPLE 6

Fixed-dose combination of 100 milligrams sitagliptin and 30 milligrams
pioglitazone/per tablet - fluid bed wet granulation

| Ingredient (mg/unit) | 100 mg/30 mg |
|---|---|
| Sitagliptin phosphate monohydrate* | 128.5 mg |
| Pioglitazone hydrochloride** (jet milled) | 33.06 mg |
| Hydroxypropylcellulose (HPC) | 12.85 mg |
| Microcrystalline cellulose (Avicel PH-102) | 74.66 mg |
| Dibasic Calcium Phosphate, anhydrous (A-Tab) | 123.75 mg |
| Crospovidone | 21.18 mg |
| 12 mg extragranular; 9.18 mg intragranular | |
| Sodium stearyl fumarate | 6.00 mg |
| Purified water for granulation step*** | 201.32 mg containing |
| | the above 12.85 mg of |
| | HPC for fluid bed |
| Total Tablet Weight | 400 mg |
| Opadry ® White 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 30 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, anhydrous dibasic calcium phosphate and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 400 mg uncoated tablet. The tablets were optionally film coated with a suitable Opadry® suspension (such as Opadry® 20A18334) to an approximate 3% weight gain to provide a 412 mg coated tablet.

### EXAMPLE 7

Fixed-dose combination of 100 milligrams sitagliptin and 45 milligrams
pioglitazone/per tablet - fluid bed wet granulation

| Ingredient (mg/unit) | 100 mg/45 mg |
|---|---|
| Sitagliptin phosphate monohydrate* | 128.5 mg |
| Pioglitazone hydrochloride** (jet milled) | 49.59 mg |
| Hydroxypropylcellulose (HPC) | 14.17 mg |
| Microcrystalline cellulose (Avicel PH-102) | 55.88 mg |
| Dibasic Calcium Phosphate, anhydrous (A-Tab) | 123.75 mg |
| Crospovidone | 22.12 mg |
| 12 mg extragranular; 10.12 mg intragranular | |
| Sodium stearyl fumarate | 6.00 mg |
| Purified water for granulation step*** | 221.99 mg containing |
| | the above 14.17 mg of |
| | HPC mg for fluid bed |
| Total Tablet Weight | 400 mg |
| Opadry ® White 20A18334 (Film Coat) | 12.0 mg |
| Purified water for coating step*** | 108 mg |
| Tablet Weight with Coating | 412 mg |

*Equivalent to 100 mg of sitagliptin free base anhydrate.
**Equivalent to 45 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, anhydrous dibasic calcium phosphate and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 400 mg uncoated tablet. The tablets were optionally film coated with a suitable Opadry® suspension (such as Opadry® 20A18334) to an approximate 3% weight gain to provide a 412 mg coated tablet.

### EXAMPLE 8

Fixed-dose combination of 50 milligrams sitagliptin and 15 milligrams
pioglitazone/per tablet - fluid bed wet granulation

| Ingredient (mg/unit) | 50 mg/15 mg |
|---|---|
| Sitagliptin phosphate monohydrate* | 64.25 mg |
| Pioglitazone hydrochloride** (jet milled) | 16.53 mg |
| Hydroxypropylcellulose (HPC) | 6.43 mg |
| Microcrystalline cellulose (Avicel PH-102) | 49.60 mg |
| Mannitol (SD 200) | 49.60 mg |
| Crospovidone | 10.59 mg |
| 6 mg extragranular; 4.59 mg intragranular | |
| Sodium stearyl fumarate | 3.00 mg |
| Purified water for granulation step*** | 100.74 mg containing |
| | the above 6.43 mg of |
| | HPC for fluid bed |
| Total Tablet Weight | 200 mg |
| Opadry ® (Film Coat) | 4 mg to 10 mg |
| Purified water for coating step*** | 36 mg to 90 mg |
| Tablet Weight with Coating | 204 mg to 210 mg |

*Equivalent to 50 mg of sitagliptin free base anhydrate.
**Equivalent to 15 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to the mixture of sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, mannitol and crospovidone were added to the granules, and blended in a twin shell-blender for 10 minutes. The lubricant sodium stearyl fumarate was then added, and the mixture was blended an additional 5 minutes. Then the lubricated mixture was compressed using a rotary tablet press to provide a 200 mg uncoated tablet. The tablets were optionally film coated with a suitable Opadry® suspension to an approximate 2-5% weight gain to provide a coated tablet weighing approximately 204 mg to 210 mg.

### EXAMPLE 9

Fixed-dose combination of 50 milligrams sitagliptin and 30 milligrams
pioglitazone/per tablet - fluid bed wet granulation

| Ingredient (mg/unit) | 50 mg/30 mg |
|---|---|
| Sitagliptin phosphate monohydrate* | 64.25 mg |
| Pioglitazone hydrochloride** (jet milled) | 33.06 mg |
| Hydroxypropylcellulose (HPC) | 7.74 mg |
| Microcrystalline cellulose (Avicel PH-102) | 64.09 mg |
| Mannitol (SD 200) | 64.09 mg |
| Crospovidone | 13.03 mg |
| 7.5 mg extragranular; 5.53 mg intragranular | |
| Sodium stearyl fumarate | 3.75 mg |
| Purified water for granulation step*** | 121.26 mg containing |
| | the above 7.74 mg of |
| | HPC for fluid bed |
| Total Tablet Weight | 250 mg |
| Opadry ® (Film Coat) | 5 mg to 12.5 mg |
| Purified water for coating step*** | 45 mg to 87.5 mg |
| Tablet Weight with Coating | 255 mg to 262.5 mg |

*Equivalent to 50 mg of sitagliptin free base anhydrate.
**Equivalent to 30 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to the mixture of sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, mannitol and crospovidone were added to the granules, and blended in a twin shell-blender for 10 minutes. The lubricant sodium stearyl fumarate was then added, and the mixture was blended an additional 5 minutes. Then the lubricated mixture was compressed using a rotary tablet press to provide a 250 mg uncoated tablet. The tablets were optionally film coated with a suitable Opadry® suspension to an approximate 2-5% weight gain to provide a coated tablet weighing approximately 255 mg to 262.5 mg.

EXAMPLE 10

Fixed-dose combination of 50 milligrams sitagliptin and 15 milligrams
pioglitazone/per tablet - fluid bed wet granulation

| Ingredient (mg/unit) | 50 mg/15 mg |
|---|---|
| Sitagliptin phosphate monohydrate* | 64.25 mg |
| Pioglitazone hydrochloride** (jet milled) | 16.53 mg |
| Hydroxypropylcellulose (HPC) | 6.43 mg |
| Microcrystalline cellulose (Avicel PH-102) | 37.33 mg |
| Dibasic Calcium Phosphate, anhydrous (A-Tab) | 61.88 mg |
| Crospovidone | 10.59 mg |
| 6 mg extragranular; 4.59 mg intragranular | |
| Sodium stearyl fumarate | 3.00 mg |
| Purified water for granulation step*** | 100.74 mg containing |
| | the above 6.43 mg of |
| | HPC for fluid bed |
| Total Tablet Weight | 200 mg |
| Opadiy ® (Film Coat) | 4 mg to 10 mg |
| Purified water for coating step*** | 36 mg to 90 mg |
| Tablet Weight with Coating | 204 mg to 210 mg |

*Equivalent to 50 mg of sitagliptin free base anhydrate.
**Equivalent to 15 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, anhydrous dibasic calcium phosphate and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 200 mg uncoated tablet. The tablets were optionally film coated with a suitable Opadry® suspension (such as Opadry® 20A18334) to an approximate 2-5% weight gain to provide a coated tablet weighing approximately 204 mg to 210 mg.

### EXAMPLE 11

Fixed-dose combination of 50 milligrams sitagliptin and 30 milligrams
pioglitazone/per tablet - fluid bed wet granulation

| Ingredient (mg/unit) | 50 mg/30 mg |
|---|---|
| Sitagliptin phosphate monohydrate* | 64.25 mg |
| Pioglitazone hydrochloride** (jet milled) | 33.06 mg |
| Hydroxypropylcellulose (HPC) | 7.74 mg |
| Microcrystalline cellulose (Avicel PH-102) | 50.83 mg |
| Dibasic Calcium Phosphate, anhydrous (A-Tab) | 77.34 mg |
| Crospovidone | 13.03 mg |
| 7.5 mg extragranular; 5.53 mg intragranular | |
| Sodium stearyl fumarate | 3.75 mg |
| Purified water for granulation step*** | 121.26 mg containing |
| | the above 7.74 mg of |
| | HPC for fluid bed |
| Total Tablet Weight | 250 mg |
| Opadry ® (Film Coat) | 5 mg to 12.5 mg |
| Purified water for coating step*** | 45 mg to 87.5 mg |
| Tablet Weight with Coating | 255 mg to 262.5 mg |

*Equivalent to 50 mg of sitagliptin free base anhydrate.
**Equivalent to 30 mg of pioglitazone free base.
***Removed during processing.

### Method of Manufacture:

Preparation of intragranular portion: Sitagliptin phosphate monohydrate, pioglitazone hydrochloride and crospovidone were loaded into a fluid bed granulator. In the case of fluid bed granulation, purified water containing hydroxypropylcellulose, was added to sitagliptin phosphate monohydrate, pioglitazone hydrochloride and crospovidone over a period of 60 to 130 minutes. The wetted mass was dried in a fluid-bed dryer at an inlet temperature of 55-75° C. The dried material was then de-lumped using a co-mill to achieve uniform granules.

Addition of the extragranular portion: After de-lumping, microcrystalline cellulose, anhydrous dibasic calcium phosphate and crospovidone were added to the granules, and the mixture was blended in a twin shell-blender for 10 minutes. The lubricant sodium stearyl fumarate was then added and the mixture was blended an additional 5 minutes. The lubricated mixture was compressed using a rotary tablet press to provide a 250 mg uncoated tablet. The tablets were optionally film coated with a suitable Opadry® suspension (such as Opadry® 20A18334) to an approximate 2-5% weight gain to provide a coated tablet weighing approximately 255 mg to 262.5 mg.

What is claimed is:

1. A pharmaceutical composition comprising:
(a) an intragranular portion comprising
(i) about 15 to 60% by weight of a dipeptidyl peptidase-4 inhibitor, or a pharmaceutically acceptable salt thereof;
(ii) about 2 to 24% by weight of pioglitazone hydrochloride; and
(iii) about 1 to 8% by weight of a binding agent; and
(b) an extragranular portion.

2. The pharmaceutical composition of claim 1 wherein the intragranular portion further comprises about 1 to 8% of a disintegrant.

3. The pharmaceutical composition of claim 1 wherein the extragranular portion comprises one or more excipients selected from the group consisting of: (a) a diluent; (b) a lubricant; and (c) a disintegrant.

4. The pharmaceutical composition of claim 1 comprising:
(a) an intragranular portion comprising:
(i) about 15 to 60% by weight of a dipeptidyl peptidase-4 inhibitor, or a pharmaceutically acceptable salt thereof;
(ii) about 2 to 24% by weight of pioglitazone hydrochloride; and
(iii) about 1 to 8% by weight of a binding agent; and
(b) an extragranular portion comprising:
(i) about 2 to 9% by weight of a disintegrant;
(ii) about 0 to 80% by weight of a diluent; and
(iii) about 0.1 to 10% by weight of a lubricant.

5. The pharmaceutical composition of claim 4 wherein the intragranular portion further comprises about 1 to 8% of a disintegrant.

6. The pharmaceutical composition of claim 4 wherein the binding agent is hydroxypropylcellulose; the disintegrant is crospovidone; the diluent is microcrystalline cellulose, mannitol or anhydrous dibasic calcium phosphate, or a mixture thereof; and the lubricant is magnesium stearate or sodium stearyl fumarate, or a mixture thereof.

7. The pharmaceutical composition of claim 4 wherein the binding agent is hydroxypropylcellulose; the disintegrant is crospovidone; the diluent is a mixture of microcrystalline cellulose and mannitol; and the lubricant is sodium stearyl fumarate.

8. The pharmaceutical composition of claim 4 wherein the binding agent is hydroxypropylcellulose; the disintegrant is crospovidone; the diluent is microcrystalline cellulose; and the lubricant is sodium stearyl fumarate.

9. The pharmaceutical composition of claim 4 wherein the binding agent is hydroxypropylcellulose; the disintegrant is crospovidone; the diluent is a mixture of microcrystalline cellulose and anhydrous dibasic calcium phosphate; and the lubricant is sodium stearyl fumarate.

10. The pharmaceutical composition of claim 4 wherein the dipeptidyl peptidase-4 inhibitor is selected from the group consisting of alogliptin, carmegliptin, denagliptin, dutogliptin, linagliptin, melogliptin, saxagliptin, sitagliptin, and vildagliptin, or a pharmaceutically acceptable salt of each thereof.

11. The pharmaceutical composition of claim 10 wherein the dipeptidyl peptidase-4 inhibitor is sitagliptin, or the dihydrogenphosphate salt thereof.

12. The pharmaceutical composition of claim 1 comprising:
(a) an intragranular portion comprising:
(i) about 21 to 33% by weight of a dipeptidyl peptidase-4 inhibitor, or a pharmaceutically acceptable salt thereof;
(ii) about 3 to 13% by weight of pioglitazone hydrochloride;
(iii) about 2 to 6% by weight of a binding agent; and
(iv) about 2 to 6% of a disintegrant; and
(b) an extragranular portion comprising:
(i) about 2 to 6% by weight of a disintegrant;
(ii) about 40 to 55% by weight of a diluent; and
(iii) about 0.5 to 4% by weight of a lubricant.

13. The pharmaceutical composition of claim 1 comprising:
(a) an intragranular portion comprising:
(i) about 21 to 33% by weight of a dipeptidyl peptidase-4 inhibitor, or a pharmaceutically acceptable salt thereof;
(ii) about 4 to 13% by weight of pioglitazone hydrochloride;
(iii) about 2 to 6% by weight of a binding agent; and
(iv) about 2 to 4% of a disintegrant; and
(b) an extragranular portion comprising:
(i) about 2 to 5% by weight of a disintegrant;
(ii) about 44 to 54% by weight of a diluent; and
(iii) about 0.5 to 4% by weight of a lubricant.

14. The pharmaceutical composition of claim 1 comprising:
(a) an intragranular portion comprising:
(i) about 21 to 33% by weight of a dipeptidyl peptidase-4 inhibitor, or a pharmaceutically acceptable salt thereof;
(ii) about 4 to 13.5% by weight of pioglitazone hydrochloride;
(iii) about 2 to 6% by weight of a binding agent; and
(iv) about 2 to 4% of a disintegrant; and
(b) an extragranular portion comprising:
(i) about 2 to 5% by weight of a disintegrant;
(ii) about 44 to 55% by weight of a diluent; and
(iii) about 0.5 to 4% by weight of a lubricant.

15. The pharmaceutical composition of claim 12 wherein the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and mannitol; and the disintegrant is crospovidone.

16. The pharmaceutical composition of claim 12 wherein the dipeptidyl peptidase-4 inhibitor is sitagliptin, or a pharmaceutically acceptable salt thereof; the lubricant is sodium stearyl fumarate; the binding agent is hydroxypropylcellulose; the diluent is a mixture of microcrystalline cellulose and anhydrous dibasic calcium phosphate; and the disintegrant is crospovidone.

17. The pharmaceutical composition of claim 12 wherein the dipeptidyl peptidase-4 inhibitor is present in a unit dosage strength of 25, 50, 75, 100, 150, or 200 milligrams, and the pioglitazone is present in a unit dosage strength of 15, 30, or 45 milligrams.

18. The pharmaceutical composition of claim 17 wherein the dipeptidyl peptidase-4 inhibitor is present in a unit dosage strength of 50 or 100 milligrams, and the pioglitazone is present in a unit dosage strength of 15, 30, or 45 milligrams.

19. The pharmaceutical composition of claim 18 wherein the dipeptidyl peptidase-4 inhibitor is sitagliptin.

20. The pharmaceutical composition of claim 19 wherein the sitagliptin is present in a unit dosage strength of 50 or 100 milligrams, and the pioglitazone is present in a unit dosage strength of 15, 30 or 45 milligrams.

21. The pharmaceutical composition of claim 1 wherein the composition is in the dosage form of a tablet.

22. A method of treating Type 2 diabetes in a human in need thereof comprising orally administering to the human a pharmaceutical composition of claim 1.

23. The pharmaceutical composition of claim 1 further comprising one or more agents selected from the group consisting of flavoring agents, colorants, and sweeteners.

24. The pharmaceutical composition of claim 1 prepared by wet granulation methods,

25. The pharmaceutical composition of claim 1 wherein the dipeptidyl peptidase-4 inhibitor is vildagliptin, or a pharmaceutically acceptable salt of each thereof.

26. The pharmaceutical composition of claim 1 wherein the dipeptidyl peptidase-4 inhibitor is saxagliptin, or a pharmaceutically acceptable salt of each thereof.

27. The pharmaceutical composition of claim 1 wherein the dipeptidyl peptidase-4 inhibitor is alogliptin, or a pharmaceutically acceptable salt of each thereof.
